(19) United States
(12) Patent Application Publication
(10) Pub. No.: US 2022/0167859 A1
(43) Pub. Date: Jun. 2, 2022

(54) SYSTEM AND METHOD FOR BLOOD PRESSURE MONITORING WITH SUBJECT AWARENESS INFORMATION

(71) Applicant: LIVEMETRIC (MEDICAL) S.A., Luxembourg (LU)

(72) Inventors: Tomer BENTZION, Tel Aviv (IL); Ori HAY, Aviel (IL); Tamar AHILEA-ANHOLT, Tel Aviv (IL)

(21) Appl. No.: 17/674,156

(22) Filed: Feb. 17, 2022

Related U.S. Application Data
(63) Continuation-in-part of application No. 16/237,899, filed on Jan. 2, 2019.
(60) Provisional application No. 62/781,743, filed on Dec. 19, 2018.

(51) Int. Cl. A61B 5/0205; A61B 5/00

(52) U.S. Cl. CPC A61B 5/02055; A61B 2562/0247; A61B 5/0022; A61B 5/0004; A61B 5/4806; A61B 5/441; A61B 5/7246; A61B 5/746; A61B 5/7264; A61B 5/7275; A61B 2562/0219; A61B 2562/0271; A61B 2562/029; A61B 2560/0242; A61B 5/6801

(57) ABSTRACT

Systems and methods for monitoring of physiological signals together with subject awareness information, including measuring and analyzing blood pressure and contextual blood pressure analysis of subjects. Systems and methods of non-invasive (optionally continuous or waveform) blood pressure measurement of subjects with sensor-derived data such as subject's activity, posture, location, place, time, etc.

### CROSS REFERENCE TO RELATED APPLICATIONS

This application is a Continuation-in-part of U.S. patent application Ser. No. 16/237,899, filed Jan. 2, 2019, which claims the benefit of priority of U.S. Provisional Patent Application No. 62/781,743, filed Dec. 19, 2018, the contents of which are all incorporated herein by reference in their entirety.

### TECHNICAL FIELD

The present disclosure generally relates to a system and method for monitoring of physiological signals together with subject awareness information.

### BACKGROUND

High blood (hypertension) pressure is a common condition in which the long-term force of the blood against the artery walls is high enough that it may eventually cause health problems, such as heart disease or stroke. Blood pressure is determined both by the amount of blood the heart pumps and the amount of resistance to blood flow in the arteries. The more blood the heart pumps and the narrower the arteries, the higher the blood pressure.

One can have high blood pressure (i.e. hypertension) for years without any symptoms. However, even without symptoms, damage to blood vessels and the heart continues. Uncontrolled high blood pressure increases the risk of serious health problems, including heart attack and stroke.

Currently, cardiovascular diseases represent a large proportion of all reported deaths globally. These diseases are considered severe and shared risk, with a majority of the burden in low- and middle-income countries. Hypertension is considered a major factor that increases the risk of heart failures or strokes, speeds up hardening of blood vessels and reduces life expectancy.

Hypertension is a chronic health condition in which the pressure exerted by the circulating blood upon the walls of blood vessels is elevated. In order to ensure appropriate circulation of blood in blood vessels, the heart of a hypertensive person must work harder than normal, which increases the risk of heart attack, stroke and cardiac failure. Eating a healthy diet and exercising, however, can significantly improve blood pressure control and decrease the risk of complications. Efficient drug treatments are also available. It is therefore important to find subjects with elevated blood pressures and monitor their blood pressure information on a regular basis.

During each heartbeat, the blood pressure varies between a maximum (i.e. systolic) and a minimum (i.e. diastolic) pressure. A traditional noninvasive way to measure blood pressure has been to use a pressurized cuff and detect the pressure levels where the blood flow starts to pulsate (i.e. cuff pressure is between the systolic and diastolic pressure) and where there is no flow at all (i.e. cuff pressure exceeds systolic pressure). It has been seen, however, that users tend to consider the measurement situations, as well as the pressurized cuff, tedious and even stressful, especially in long-term monitoring.

The use of wearable devices for monitoring body physiological parameters (e.g. blood pressure, heart rate (HR) pulse, body temperature, blood glucose level, movement patterns, etc.) noninvasively, beat-to-beat, continuously and/or intermittently for extended periods of time are thus becoming popular as a way to monitor and improve health.

Traditional blood pressure measurements require inflatable cuffs, which are gradually deflated from a state of full vessel occlusion to a lower pressure while listening using a mechanical sensor (e.g., stethoscope) to the sounds generated by the blood flow eddies in the vessel. An advantage of this method is its relative robustness to arm motion, while a disadvantage is its large form factor and the need for either manual inflation by the user or an automatic pump, which requires large quantities of energy. Since energy efficiency and small form factor are major requirements in wearable devices, inflatable cuff blood pressure sensing is not a useful paradigm in this space.

In addition, blood pressure is known to be affected by the mental/emotional state of the subject, for example, the well-known white-coat syndrome tends to elevate the blood pressure during the measurement which leads to inaccurate diagnoses. There is thus a need in the art for more compliable and accurate systems and methods for blood pressure monitoring.

### SUMMARY

There are provided herein, according to some embodiments, a system and method for measurement and monitoring of physiological signals together with subject awareness information. More specifically, a system and method of non-invasive (optionally continuous or waveform) blood pressure measurement with sensor-derived data such as subject's activity, posture, location, place, time, etc. According to some embodiments, the system and method disclosed herein, rely on direct pressure sensing of one or more of the radial, ulnar or brachial arteries on the wrist or hand of the subject. Pressure sensing data is obtained by placing at least one pressure sensitive sensor upon the artery, such as radial, ulnar and/or brachial, femoral, popliteal, tibial, and/or fibular artery. In some embodiments, the pressure sensitive sensor may be comprised in a wearable device. The pressure sensed is related to the blood pressure in the arteries and may generally be referred to as a blood pressure waveform. Furthermore, in accordance with some embodiments, the system/method may include a computation component that, using special algorithms, calculates the exact blood pressure values (Systolic, Diastolic, Mean and momentary arterial blood pressure). Furthermore, in accordance with some embodiments, the system/method may include a computation component that, using special algorithms, calculates the exact intermittent blood pressure values, continuous blood pressure values (which means measuring systolic and diastolic blood pressure values once every specific period—e.g., every about 3, 5 or 10 seconds), beat-to-beat values (once every heart beat), or momentary values (also called the blood pressure waveform, i.e., “graph” values). In some embodiments, the computation component is physically associated with the system. In some embodiments, at least a portion of the computation component is remotely associated with the system (i.e., mobile service, server or cloud based). According to some embodiments, the system may incorporate additional physiological data and/or sensors such as heart rate, ECG waveform, body temperature, SpO₂, respiration rate, and/or perspiration. In some embodiments, the additional sensors are comprised in the wearable device. According to some embodiments, the system may incorporate subject awareness data, which may be obtained, for example, from sensors such as accelerometer, gyroscope, magnetometer (compass), steps counter, GPS, barometer, temperature, ambient light sensor (light level), microphone (noise level and speech recognition) which may provide combined and extrapolated subject situations such as: subject's activity (e.g. walking, running, biking, and length of), orientation and posture (standing, lying down), altitude, location (longitude and latitude), place (address, type—e.g., park, coffee shop, home, office-specific site—e.g. Hilton Hotel NY), weather, local time, environment (e.g. noisy, quiet).

### Subject Awareness can Increase Accuracy of Blood Pressure Values

Measurement of Blood pressure usually follows specific guidelines. These guidelines may have variations in different regions (e.g. Europe, United States, Japan) due to cultural and physiological differences. The guidelines for blood pressure measurements involve several aspects, including:
Subject's posture—subject's body position can significantly affect the measurement outcome. Some effects are physical in nature (e.g., raising or lowering the arm) and some are physiological in nature (e.g., sitting with legs crossed)
Subject's rest before and during measurement—reduce effects of current and previous subject activity on the blood pressure measurement. This includes avoiding physical activity prior to taking a measurement, not speaking while the measurement is in process, avoiding noisy environment, lack of proper sleep.
Subject's food and beverage intake—prohibit/avoid foods and beverages that are known to modify blood pressure (such as caffeine).
Subject's physiology—empty bladder, not smoking nicotine or intake of other substances known to modify blood pressure (other than regular medication)

The guidelines may differ in regard of specific time before measurement (timing), specific substances more common to various regions, etc. These changes can be due to different cultural habits, physiological difference due to ethnicity, etc. Furthermore, some guidelines have slightly different measurement protocol that introduce additional changes (such as changes to activity detection). For example, the Blood Pressure measurement guidelines of the American College of Cardiology (ACC) and American Heart Association (AHA) require that the subject (undergoing blood pressure measurement, e.g., a patient) should be relaxed, sitting in a chair for more than 5 minutes. The subject should avoid caffeine, exercise, and smoking for at least 30 minutes before measurement. Neither the subject nor persons in his surroundings should talk during the rest period or during the measurement. Measurements made while the subject is lying on an examining table do not fulfill these criteria. Whelton, Paul K., et al. “2017 ACC/AHA/AAPA/ABC/ACPM/AGS/APhA/ASH/ASPC/NMA/PCNA guideline for the prevention, detection, evaluation, and management of high blood pressure in adults: a report of the American College of Cardiology/American Heart Association Task Force on Clinical Practice Guidelines.” Journal of the American College of Cardiology 71.19 (2018): e127-e248.

Another example of guidelines with different rules specifics, the European Society of Cardiology/European Society of Hypertension (ESC/ESH) Guidelines simply states that home measurement should be taken in a quiet room after 5 min of rest, with the patient seated with their back and arm supported. For office measurement it also incudes that in older people, people with diabetes, or people with other causes of orthostatic hypotension, BP should also be measured 1 min and 3 min after standing (i.e. the patient should stand than sit down and then measure their BP). Williams, Bryan, et al. “2018 ESC/ESH Guidelines for the management of arterial hypertension: The Task Force for the management of arterial hypertension of the European Society of Cardiology (ESC) and the European Society of Hypertension (ESH).” European heart journal 39.33 (2018): 3021-3104.

Current devices are not aware of the subject activity and cannot validate or disqualify a measurement. The few devices that do measure blood pressure over the whole day (e.g. Holter) usually ignore measurements taken while the subject is moving.

Advantageously, applying subject awareness information/data can be useful in at least two ways—it can validate the measurement (for example, in accordance with the guidelines) and it can also adjust measurement values when the measurement is taken in conditions that do not comply with the guidelines.

The systems and methods disclosed herein, in accordance with some embodiments, may identify the subject's posture and orientation, e.g., by using motion and orientation sensors, and confirm that the subject is sitting before and while the measurement is carried out. The system may also identify prior activity (e.g., exercise or excessive physical activity) for example, by using motion and orientation sensors or analyzing heart rate changes over time by using ECG and/or blood pressure sensors. The system may identify a “noisy” environment—in terms of sound and/or light level, as well as identify talking while the measurement is taking place, by using a microphone and/or ambient light sensor. Thus, user awareness can validate blood pressure measurement in accordance with the guidelines.

In some exemplary embodiments, the use of awareness data can provide information regarding subject's posture, rest (or activity), talking, or even about intake of food and beverage (by analyzing wrist motion related to eating and drinking).

According to some embodiments, awareness data includes subject activity tracking. The system may record current and historical activity information (current and past activity) in order to provide information regarding subject's rest before and during measurement. This may include, for example, sleep tracking, motion tracking, monitoring of speech and noise.

In some embodiments, this can be done using sensor fusion and data analysis using machine learning or expert systems. For example, a machine learning model (e.g. random forest or neural network) can be trained to identify wrist movement during eating for example, from accelerometer and gyroscope data within a wearable measuring device (wristband)). Such machine learning model can be general (i.e., model trained based on large population data), subject specific (i.e., model trained on data obtained from the specific subject), or hybrid (i.e., initially use general model, but add subject specific data to “adjust” or fine-tune the model). Another example make use of a microphone to analyze the background noise and detect speech. Such speech detection can be based on machine learning with the sound data given as input. The model used for speech detection may be trained on large population data (a general model) or a model trained to identify the voice of the specific subject and detect only when the subject is speaking (and not any speech sound in the background).

According to some embodiments, the system may also be able to compensate for various situations differing from those defined in the guidelines, so the measurements, for example, while sleeping (lying down) or after exercise, could be used for blood pressure monitoring. According to some embodiments, the system may use previous recorded data (either of the same user or of a large population) to associate BP values measured according to the guidelines with values measured just after specific conditions have changed (e.g., the BP values while the subject is talking, or shortly after physical activity), and use the association to adjust BP values deviating from the guidelines to BP values according to the guidelines.

According to additional or alternative embodiments, recorded values, e.g., blood pressure values, which were measured in a different setup from the guidelines, could be adjusted to correlate to guideline measurement using subject awareness information. For example, high values during exercise or low values during deep sleep could be correlated with corresponding (lower or higher) values that would be measured according to the guidelines, using subject awareness information. This information may be used to identify the activity, the (short term) history, and even to (learn and) create a subject specific adjustment function. This will allow the subject/caregiver/clinician to have a full blood pressure profile and assist in identifying root causes for hypertension and other blood pressure related conditions.

In some embodiments, the system can use the measurement results together with the awareness information to provide an additional result that correlates to measurement done according to the guidelines. This is important since it can provide clinicians with 24/7 monitoring of blood pressure in normative terms (i.e. as measurement according to the standard). For example, such adjusted data can assist with better assessment of blood pressure variability, which is considered as high risk as hypertension. Without adjustment, the blood pressure variability of very active subjects such as physical workers would be high (compared to less active population), while adjusted values would allow more precise comparison to the general (less active) population and identify subjects with true blood pressure variability.

According to some embodiments, adjustment of blood pressure values requires combining identification of the current state of the subject with a method/calculation/or function that can adjust (or transfer or translate) these BP values to corresponding BP values taken as if the subject state was according to the guidelines. In some embodiments, the BP values adjustment may be done using methods/calculations/functions based on one or more of the following: physiological modeling, statistical methods (e.g., statistical correlation), use of machine learning, or combinations thereof.

According to some embodiments, any of the methods for adjustment of BP values can be either: generic (e.g., based on large population data (and not specific to the current subject)); subject specific (e.g., configured using data obtained from a specific subject); or hybrid (e.g. initially use generic configuration, and using subject specific data to “adjust” or fine-tune the method or function).

According to some embodiments, the BP values adjustment may be performed using physiological modeling. According to some embodiments, the physiological model may include, for example, but not limited to, any one or more of a Windkessel model, one or more 0D models, one or more one-dimensional (1D) models, or any combination thereof. According to some embodiments, the physiological model may include implementing one or more models of physiological mechanisms and/or analyses associated with how a specific subject activity or condition is supposed to affect the arterial pressure waveform. According to some embodiments, the physiological model may include implementing one or more models of physiological mechanisms and/or analyses associated with inversing the effect of specific subject activity or condition on the arterial pressure waveform. According to some embodiments, BP values adjustment may be carried out using the physiological model together with the arterial pressure waveform and the subject status and/or activity to inverse the effect of subjects' condition or activity.

According to some embodiments the BP values adjustment may be performed using statistical methods, such as correlating unvalidated measurements to validated subject measurements just prior and/or post to the unvalidated measurements. According to some embodiments, the correlation can be simple linear correlation or a more complex multi-dimensional statistical correlation that also takes into account subject's status and activity.

According to some embodiments, the BP values adjustment may be performed using machine learning algorithm (or in other words, machine learning model). According to some embodiments, the machine learning algorithm may include any one or more of regression models, neural networks, (deep) convolutional networks, support vector regressor, or any combination thereof. According to some embodiments, the machine learning model may be trained to inverse the effects of subject's status or activity on the BP values or arterial waveform.

### Increase Blood Pressure Monitoring Information Using Subject Awareness

Blood pressure, along with various other physiological signals, is greatly influenced by the state of the subject such as: current activity, time of day, feelings, energy etc. Advantageously, combining (momentary) blood pressure measurement with subject awareness parameters allows for more accurate clinical diagnosis. Advantageously, combining subject awareness with blood pressure information facilitates identification of the causes of high blood pressure, for example, due to stressful situations (e.g., driving in heavy traffic), activity (e.g., exercise), or time of day (e.g., lunch time). Advantageously, the system can then use subject activity information, for example, to examine how various activities affect the subject (e.g. sleeping vs. walking vs. sitting still). The system may also compare the blood pressure information in various locations (e.g. at home vs. office vs. on the road), or time of day. The additional information can enhance the simple blood pressure measurement and provide context to various changes in the subject that a caregiver/clinician may see within the physiological data. The additional information may allow clinicians to differentiate between high BP values measured with apparent context (e.g. stressful situation, lack of sleep, noisy environment) and values measured with “ordinary” context. The additional information may allow clinicians to disregard measurements taken in stressful situations or locations. Advantageously, this allows the subject/caregiver/clinician to have a full blood pressure profile and assist in identifying root causes for hypertension and other blood pressure related conditions.

### Diagnosis of Blood Pressure Disorders Based on Subject Awareness

In accordance with some embodiments, blood pressure disorders such as primary and secondary hypertension, hypotension, and fluctuating blood pressure may be diagnosed more accurately when combining blood pressure measurement over period of time and subject awareness parameters. For example, white coat syndrome may easily be diagnosed and distinguished from hypertension by taking blood pressure measurement throughout the day with subject awareness information—specifically geolocation, place, and activity—and detecting if high blood pressure values occur when the measurements take place at specific places (e.g. hospital, clinic, kiosk) or are consistent throughout the day. Another example is diagnosis of secondary hypertension induced by obstructive sleep apnea by identifying sleep in general and sleep patterns using activity detection (e.g., using accelerometer, gyro and magnetometer together with ambient light sensor) together with heart rate or breathing rate detection, e.g., using PPG (photoplethysmography), ECG, or blood pressure sensor. Combining blood pressure measurement with subject awareness parameters may also facilitate diagnosing highly variable blood pressure by identifying fluctuating blood pressure and differentiating it from normal fluctuations. Normally, blood pressure values fluctuate throughout the day, and often large fluctuations of blood pressure values may occur, but for caregivers/clinicians it is difficult to differentiate fluctuating blood pressure syndrome from normal fluctuations because of changing activities (e.g., measurements taken while exercising compared to resting afterwards). In accordance with some embodiments, the method/system/device disclosed herein, capable of providing subject awareness information alongside blood pressure measurements, offers caregivers/clinicians a simple method for diagnoses of various blood pressure disorders by correlating measured values to the status of the subject (for example, the subject's activity, posture, location, place, time, etc.) at the time of measurement.

Alerts Using Blood Pressure Monitoring with Subject Awareness

In accordance with some embodiments, the method/system/device for blood pressure monitoring disclosed herein may further be configured to alert subjects of situations where their blood pressure values are beyond acceptable or normal range. The method/system/device for blood pressure monitoring may further include alerting users/subjects before the blood pressure values exceed the acceptable or normal range, by predicting future blood pressure values or trends, thus preventing dangerously high or low blood pressure values. The prediction may be subject specific (i.e., based on past/present information of the user) or generic (based on information from a general population or sub-population having similar demographics/characteristics) or a combination of both. The analysis may include current and/or past user states, where user state may include physiological measurements, subject awareness information and subject specific demographics. For example, the monitoring device, in accordance with some embodiments, may be configured to identify a situation where being at the office at a specific time where blood pressure values are usually somewhat elevated, might be too stressful when combined with lack of sleep the previous night, and lack of exercise the previous week. In accordance with some embodiments, the method/system/device for blood pressure monitoring disclosed herein, may further learn and/or correlate stressful locations and times (e.g., by recording blood pressure values with location and time) and combine it with user state that can be identified using subject awareness (e.g. identifying {lack of} sleep by using activity recognition and observing that the user slept 4 hours last night). Thus, this monitoring system can not only record and monitor blood pressure but also actively alert for hazardous situations.

There is provided herein, in accordance with some embodiments, a system for measuring blood pressure of a subject, the system includes: a pressure sensor configured to sense pressure at a peripheral artery of the subject and to provide a signal representing a waveform of the blood pressure; and electric circuitry and associated software/firmware/computation component/algorithm configured to: compute one or more blood pressure values and/or blood pressure related values based on the signal representing a waveform of the blood pressure; obtain, from one or more subject awareness sensors and/or medical or non-medical user sources, signal(s) indicative of one or more subject awareness parameters and/or one or more physiologic parameters of the subject; and validate the one or more blood pressure values by determining whether the one or more subject awareness parameters and/or the one or more physiologic parameters of the subject comply with blood pressure measurement rules.

According to some embodiments, the electric circuitry and associated software/firmware/computation component/algorithm are further configured to adjust the one or more computed blood pressure values and/or blood pressure related values to comply with blood pressure measurement rules, if at least one of the one or more subject awareness parameters and/or the one or more physiologic parameters of the subject does not comply with the rules.

There is further provided herein, in accordance with some embodiments, a system for contextual blood pressure analysis, the system includes: a wearable pressure sensor configured to measure directly/sense pressure at a peripheral artery of the subject and to provide a signal representing a waveform of the blood pressure; and electric circuitry and associated software/firmware/computation component/algorithm configured to: compute one or more blood pressure values and/or blood pressure related values based on the signal representing a waveform of the blood pressure; obtain, from one or more subject awareness sensors and/or medical or non-medical user sources, signal(s) indicative of one or more subject awareness parameters and/or one or more physiologic parameters of the subject; analyze the one or more computed blood pressure values and/or blood pressure related values with the one or more subject awareness parameters and/or the one or more physiologic parameters; and provide contextual blood pressure data.

There is further provided herein, in accordance with some embodiments, a method for measuring blood pressure of a subject, the method includes: obtaining, from a pressure sensor, a signal representing a waveform of the blood pressure of the subject; computing one or more blood pressure values and/or blood pressure related values; obtaining, from one or more subject awareness sensors and/or medical or non-medical user sources, signal(s) indicative of one or more subject awareness parameters and/or one or more physiologic parameters of the subject; and validating the one or more blood pressure values by determining whether the one or more subject awareness parameters and/or the one or more physiologic parameters of the subject comply with blood pressure measurement rules.

The method may further include adjusting the one or more computed blood pressure values and/or blood pressure related values to comply with the blood pressure measurement rules, if at least one of the one or more subject awareness parameters and/or the one or more physiologic parameters of the subject does not comply with the rules.

The method may further include measuring the one or more subject awareness parameters, utilizing the one or more subject awareness sensors, before, during and/or after measuring the blood pressure waveform utilizing the pressure sensor.

The method may further include measuring the one or more physiologic parameters of the subject, utilizing one or more sensors, before, during and/or after measuring the blood pressure waveform.

According to some embodiments, the one or more computed blood pressure values may include Systolic, Diastolic, Mean, momentary arterial blood pressure or any combination thereof.

According to some embodiments, the one or more computed blood pressure related values may include heart rate and/or breathing rate.

According to some embodiments, the one or more subject awareness sensors may include accelerometer, gyroscope, magnetometer (compass), steps counter, GPS, barometer, temperature sensor, ambient light sensor (light level), microphone (noise level and speech recognition), humidity sensor, impedance sensor or any combination thereof.

According to some embodiments, the one or more subject awareness parameters may include one or more parameters related to the subject's present and/or past (historic) surrounding.

According to some embodiments, the one or more subject awareness parameters related to the subject's present and/or past (historic) surroundings may include altitude, location, place, weather, local time, light level, surrounding noise type and/or level, level of crowdedness, traffic status or any combination thereof.

According to some embodiments, the one or more physiologic parameters may include one or more present and/or past (historic) physiologic parameters selected from the group consisting of: the subject's activity and/or length/intensity thereof, orientation, posture, sleep vs. awake, heart rate, respiration rate, skin humidity/sweat level, or any combination thereof.

According to some embodiments, the one or more medical and non-medical user sources may include a health App, a social platform, a calendar, a fitness App, a communication App or any combination thereof.

According to some embodiments, the blood pressure measurement rules may include blood pressure regulatory guidelines. The blood pressure measurement rules may include awake and sleep rules. The blood pressure measurement rules may include temporal rules. The blood pressure measurement rules may include spatial and/or geographic rules.

There is further provided herein, in accordance with some embodiments, a method for contextual blood pressure analysis, the method includes: obtaining, from a pressure sensor, a signal representing a waveform of the blood pressure of the subject; computing one or more blood pressure values and/or blood pressure related values; obtaining, from one or more subject awareness sensors and/or medical or non-medical user sources, signal(s) indicative of one or more subject awareness parameters and/or one or more physiologic parameters of the subject; analyzing the one or more computed blood pressure values and/or blood pressure related values with the one or more subject awareness parameters and/or the one or more physiologic parameters; and providing contextual blood pressure data. The contextual blood pressure data may include data indicative of the variability level of the blood pressure values. The contextual blood pressure data may include a circadian pattern of blood pressure values along with respective subject awareness parameters.

The method may further include identifying one or more correlations between the blood pressure values and the one or more subject awareness parameters, for example, correlation between high blood pressure and length of sleep in the previous night, or normal blood pressure (no hypertension) when doing physical activity on the same day or day before.

The method may further include, providing, based on the one or more correlations, a diagnosis related to blood pressure, cardiac activity and/or related disorder, for example, high blood pressure, high blood pressure variability, white coat syndrome, sleep apnea, aortic valve regurgitation (Pulsus bisferiens), Pulsus alternans and/or left ventricular impairment, Pulsus paradoxus, and Pre-eclampsia.

The method may further include, based on the one or more correlations, identifying a hazardous situation.

The method may further include, providing a blood pressure alert prior to initiation of the hazardous situation.

The method may further include, utilizing machine learning algorithms, learning one or more of the subject's habits based on the one or more correlations, and predicting the subject's blood pressure behavior in a defined situation.

The method may further include, measuring the one or more subject awareness parameters, utilizing the one or more subject awareness sensors, before, during and/or after measuring the blood pressure waveform utilizing the pressure sensor.

The one or more computed blood pressure values may include Systolic, Diastolic, Mean, momentary arterial blood pressure or any combination thereof. The one or more computed blood pressure related values may include heart rate and/or breathing rate. The one or more subject awareness sensors may include accelerometer, gyroscope, magnetometer (compass), steps counter, GPS, barometer, temperature sensor, ambient light sensor (light level), microphone (noise level and speech recognition), humidity sensor, impedance sensor or any combination thereof.

According to some embodiments, the one or more subject awareness parameters may include one or more parameters related to the subject's present and/or past (historic) surrounding. The one or more subject awareness parameters related to the subject's present and/or past (historic) surroundings may include altitude, location, place, weather, local time, light level, surrounding noise type and/or level, level of crowdedness, traffic status or any combination thereof.

According to some embodiments, the one or more physiologic parameters may include one or more present and/or past (historic) physiologic parameters selected from the group consisting of: the subject's activity and/or length/intensity thereof, orientation, posture, sleep vs. awake, heart rate, respiration rate, skin humidity/sweat level, or any combination thereof.

According to some embodiments, the one or more medical and non-medical user sources may include health Apps, social platforms, calendars, fitness Apps, communication Apps or any combination thereof.

According to some embodiments, the pressure sensor is configured to directly sense pressure at a peripheral artery, (such as radial, ulnar and/or brachial artery for the arm and femoral, popliteal, tibial, and/or fibular artery of the leg) of the subject.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. It is intended that the embodiments and figures disclosed herein are to be considered illustrative rather than restrictive. The figures are listed below:

FIG. 1 schematically depicts a block diagram of a system for monitoring blood pressure with subject awareness information, according to an exemplary embodiment of the current invention;

FIG. 2 schematically depicts a block diagram of a system for monitoring and analyzing blood pressure with subject awareness information, according to an exemplary embodiment of the current invention;

FIG. 3 schematically depicts a block diagram of a device for monitoring blood pressure with subject awareness information, the device is operable by a mobile application, according to an exemplary embodiment of the current invention;

FIG. 4 schematically depicts a flow chart of a method for monitoring blood pressure with subject awareness information, according to an exemplary embodiment of the current invention;

FIG. 5 schematically depicts a flow chart of a method for monitoring, analyzing and diagnosing a blood pressure related condition, according to an exemplary embodiment of the current invention; and

FIG. 6 schematically depicts a flow chart of a method for monitoring, analyzing and predicting a blood pressure related condition, according to an exemplary embodiment of the current invention.

### DETAILED DESCRIPTION

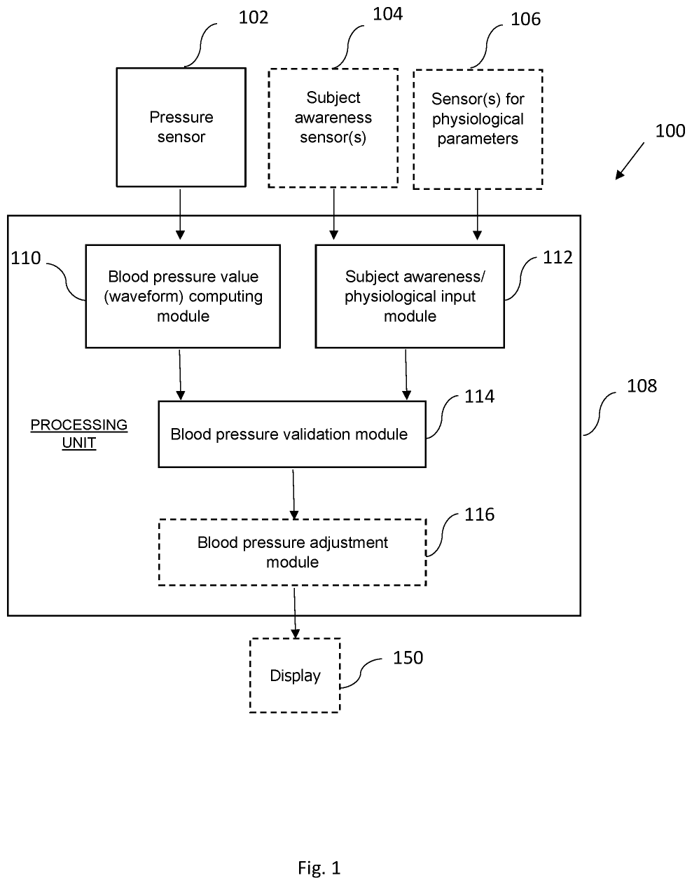

Reference is now made to FIG. 1, which schematically depicts a block diagram of a system 100 for monitoring blood pressure with subject awareness information, according to an exemplary embodiment of the current invention. System 100 includes a pressure sensor 102 which is configured to directly sense pressure at a peripheral artery, such as at a radial, ulnar and/or brachial artery for the arm and femoral, popliteal, tibial, and/or fibular artery of the leg of the subject being monitored. A signal indicative of pressure is transferred from pressure sensor 102 to a processing unit 108 and specifically to a blood pressure value (waveform) computing module 110 where blood pressure values (such as systolic, diastolic, mean or blood pressure waveform) are computed.

System 100 further includes one or more subject awareness sensors 104 and one or more physiological parameters sensors 106. Subject awareness sensor(s) 104 is configured to provide signal(s) indicative of the user awareness. More specifically, awareness sensor(s) 104 is configured to provide any type of signal indicative of the user's surroundings which may directly or indirectly affect the user's condition, well-being, state of mind, etc. For example, the user awareness signal may relate to the geolocation, place, activity, weather, local time, light level, surrounding noise type and/or level, level of crowdedness, and traffic status in the vicinity of the subject. Subject awareness sensor(s) 104 may include accelerometer, gyroscope, magnetometer (compass), steps counter, GPS, barometer, temperature sensor, ambient light sensor (light level), microphone (noise level and speech recognition), humidity sensor, impedance sensor or any combination thereof.

Physiological parameters sensor(s) 106 is configured to provide signal(s) indicative of physiologic data of the user. Such data may include heart rate, ECG waveform, EEG waveform, body temperature, SpO₂, EtCO₂, respiration rate, blood glucose level, etc.

Signal(s) received from subject awareness sensor(s) 104 and/or from physiological parameters sensor(s) 106 are transmitted to processing unit 108 and specifically to a subject awareness/physiological input module 112 to produce subject awareness/physiological parameters data from the received signals.

Data from blood pressure value (waveform) computing module 110 and from subject awareness/physiological input module 112 is transmitted to blood pressure validation module 114 of processing unit 108.

Blood pressure validation module 114 is configured to apply a set of predetermined rules to the data provided from subject awareness/physiological input module 112 and thus to determine whether blood pressure values (such as waveform) received from blood pressure value (waveform) computing module 110 can be validated. The predetermined rules may include, for example, guidelines (such as regulatory guidelines, blood pressure monitoring device manufacturer guidelines, etc.) that define the environmental/physiological conditions the subject needs to experience in order to provide accurate and reliable blood pressure values.

If the blood pressure value(s) (such as waveform) comply with predetermined rules, the blood pressure value(s) are validated. If, on the other hand, the blood pressure value(s) (such as waveform) do not comply with predetermined rules, the subject may be asked to correct the external conditions and repeat the measurement.

Furthermore, if the blood pressure value(s) (such as waveform) do not comply with predetermined rules, the blood pressure value(s) may be adjusted accordingly by a blood pressure adjustment module 116. Blood pressure value(s), whether validated or adjusted, may be displayed on a display 150, which may be any type of display, visual, vocal and or tactile, such as a computer, mobile device, watch or any other display.

According to some embodiments, the blood pressure adjustment module 116 may utilize methods for adjustment of blood pressure values based on combining identification of the state of the subject with a method (or function) that can adjust (or transfer or translate) these BP values to corresponding BP values taken as if the subject state was according to the guidelines. In some embodiments, the BP values adjustment is done using methods or functions based on one or more of the following: physiological modeling, statistical methods (e.g., statistical correlation), and use of machine learning.

According to some embodiments, such methods for adjustment of blood pressure values may require combining the identification of the current state of the subject with a method (calculation/function) that can adjust/transfer/translate) the BP values to corresponding BP values that would have been taken as if the subject state was according to the guidelines. In some embodiments, the BP values adjustment may be done using methods or functions, based on one or more of: physiological modeling, statistical methods (e.g., statistical correlation), use of machine learning, and the like, or any combination thereof.

According to some embodiments, the methods for adjustment of BP values can be generic (based on large population data and not specific to the subject)), subject specific (configured using data obtained from the specific subject), or hybrid—(utilizing both generic configuration, as well as subject specific data to “adjust” or fine-tune the method or function).

According to some embodiments, the BP values adjustment may be done using physiological modeling. In some embodiments, the physiological model may include, but is not limited to, any one or more of a Windkessel model, one or more 0D models, one or more one-dimensional (1D) models, and the like, or any combination thereof. According to some embodiments, the physiological model may include implementing one or more models of physiological mechanisms and/or analyses associated with how a specific subject activity or condition is expected to affect the arterial pressure waveform. According to some embodiments, the physiological model may include implementing one or more models of physiological mechanisms and/or analyses associated with inversing the effect of specific subject activity or condition on the arterial pressure waveform. According to some embodiments BP values adjustment may be carried out using the physiological model together with the arterial pressure waveform and the subject status and/or activity to inverse the effect of subjects' condition or activity.

According to some embodiments, the BP values adjustment may be done using statistical methods, such as correlating unvalidated measurements to validated subject measurements just prior and/or post to the unvalidated measurements. According to some embodiments the correlation can be simple linear correlation or a multi-dimensional statistical correlation that also takes into account the subject's status and activity.

According to some embodiments the BP values adjustment may be done using machine learning algorithm/model. According to some embodiments, the machine learning algorithm may include any one or more of one regression models, neural networks, (deep) convolutional networks, support vector regressor, or any combination thereof. According to some embodiments, the machine learning model may be trained to inverse the effects of the subject's status or activity on the BP values or arterial waveform.

Although processing unit 108 is described in FIG. 1 as including blood pressure value (waveform) computing module 110, subject awareness/physiological input module 112, blood pressure validation module 114, and optionally, blood pressure adjustment module 116, it is noted that these modules may be combined in one processing unit or may be separated. For example, some of these modules may be part of a blood pressure monitoring device or an app related thereto or may be remotely present, such as in a remote server (cloud).

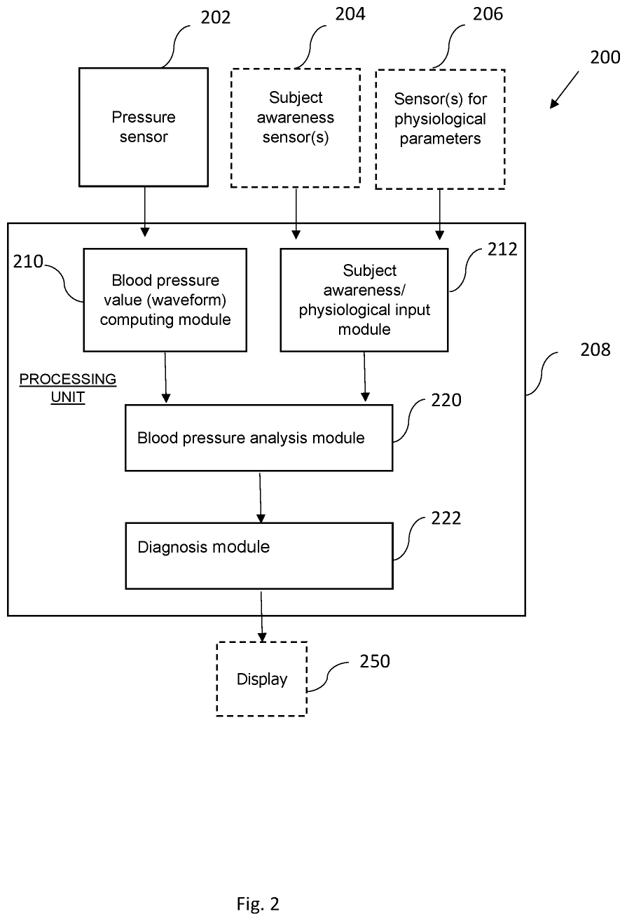

Reference is now made to FIG. 2, which schematically depicts a block diagram of a system 200 for monitoring and analyzing blood pressure with subject awareness information, according to an exemplary embodiment of the current invention. System 200 includes a pressure sensor 202 which is configured to directly sense pressure at a peripheral artery, such as at a radial, ulnar and/or brachial artery for the arm and femoral, popliteal, tibial, and/or fibular artery of the leg of the subject being monitored. A signal indicative of pressure is transferred from pressure sensor 202 to a processing unit 208 and specifically to a blood pressure value (waveform) computing module 210 where blood pressure values (such as blood pressure waveform) are computed.

System 200 further includes one or more subject awareness sensors 204 and one or more physiological parameters sensors 206. Subject awareness sensor(s) 204 is configured to provide signal(s) indicative of the user awareness. More specifically, awareness sensor(s) 204 is configured to provide any type of signal indicative of the user's surroundings which may directly or indirectly affect the user's condition, well-being, state of mind, etc. For example, the user awareness signal may relate to the geolocation, place, activity, weather, local time, light level, surrounding noise type and/or level, level of crowdedness, and traffic status in the vicinity of the subject. Subject awareness sensor(s) 204 may include accelerometer, gyroscope, magnetometer (compass), steps counter, GPS, barometer, temperature sensor, ambient light sensor (light level), microphone (noise level and speech recognition), humidity sensor, impedance sensor or any combination thereof.

Physiological parameters sensor(s) 206 is configured to provide signal(s) indicative of physiologic data of the user. Such data may include heart rate, ECG waveform, EEG waveform, body temperature, SpO₂, EtCO₂, respiration rate, blood glucose level, etc.

Signal(s) received from subject awareness sensor(s) 204 and/or from physiological parameters sensor(s) 206 are transmitted to processing unit 208 and specifically to a subject awareness/physiological input module 212 to produce subject awareness/physiological parameters data from the received signals.

Data from blood pressure value (waveform) computing module 210 and from subject awareness/physiological input module 212 is transmitted to a blood pressure analysis module 220 of processing unit 208. Blood pressure analysis module 220 is configured to analyze the computed blood pressure values received from blood pressure value (waveform) computing module 210 together with the subject awareness parameters and/or the one or more physiologic parameters received from subject awareness/physiological input module 212 and to provide contextual blood pressure data. According to some embodiments, the term “contextual blood pressure data” may refer to data which includes both blood pressure values and subject awareness data (as well as additional physiological data). In other words, contextual blood pressure data correlates a blood pressure value (e.g., waveform) with one or more awareness/physiological parameter that the subject is/was experiencing during or before blood pressure measurements took place, which may affect the measurements. For example, contextual blood pressure data may include correlation between blood pressure measured values and the subject's current/past activity, time of day, surroundings e.g., altitude, location, place, weather, local time, light level, noise type/level, level of crowdedness, traffic status, etc. As another example, contextual blood pressure data may point to a correlation between high blood pressure and length of sleep the previous night, or normal blood pressure (no hypertension) when doing physical activity on the same day or the day before.

According to some embodiment the blood pressure analysis module 220 may be configured to execute methods for adjustment of blood pressure values (such as described for module 116). In some embodiments, the BP values adjustment may be done using methods/calculations/functions based on one or more of: physiological modeling, statistical methods (e.g., statistical correlation), and machine learning tools.

Contextual blood pressure data provided by blood pressure analysis module 220 may then be applied by a diagnosis module 222 to determine a diagnosis related to blood pressure, cardiac activity and/or related disorder. Since such diagnosis is based on contextual blood pressure data, it is more reliable than a diagnosis obtained without such data. For example, subject monitoring showing high blood pressure variability throughout the day can either be the effect of activity (e.g., running) or true high blood pressure variability which cannot be differentiated without contextual blood pressure data. Other conditions such as white coat syndrome, sleep apnea, aortic valve regurgitation (Pulsus bisferiens), Pulsus alternans and/or left ventricular impairment, Pulsus paradoxus, and Pre-eclampsia may also be reliably and accurately diagnosed.

Blood pressure analysis module 220 may also utilize machine learning algorithms, to learn about the subject's habits based on the one or more correlations and predict the subject's blood pressure behavior in a defined situation. Blood pressure analysis module 220 may trigger an alarm prior to initiation of a situation which may affect the blood pressure of the subject in a hazardous way.

The determined blood pressure related diagnosis and/or an alert prior to initiation of the hazardous situation may be displayed on a display 250, which may be any type of display, visual, vocal and or tactile, such as a computer, mobile device, watch or any other display.

Although processing unit 208 is described in FIG. 2 as including blood pressure value (waveform) computing module 210, subject awareness/physiological input module 212, blood pressure analysis module 220 and diagnosis module 222, it is noted that these modules may be combined in one processing unit or may be separated. For example, some of these modules may be part of a blood pressure monitoring device or an app related thereto or may be remotely present, such as in a remote server (cloud).

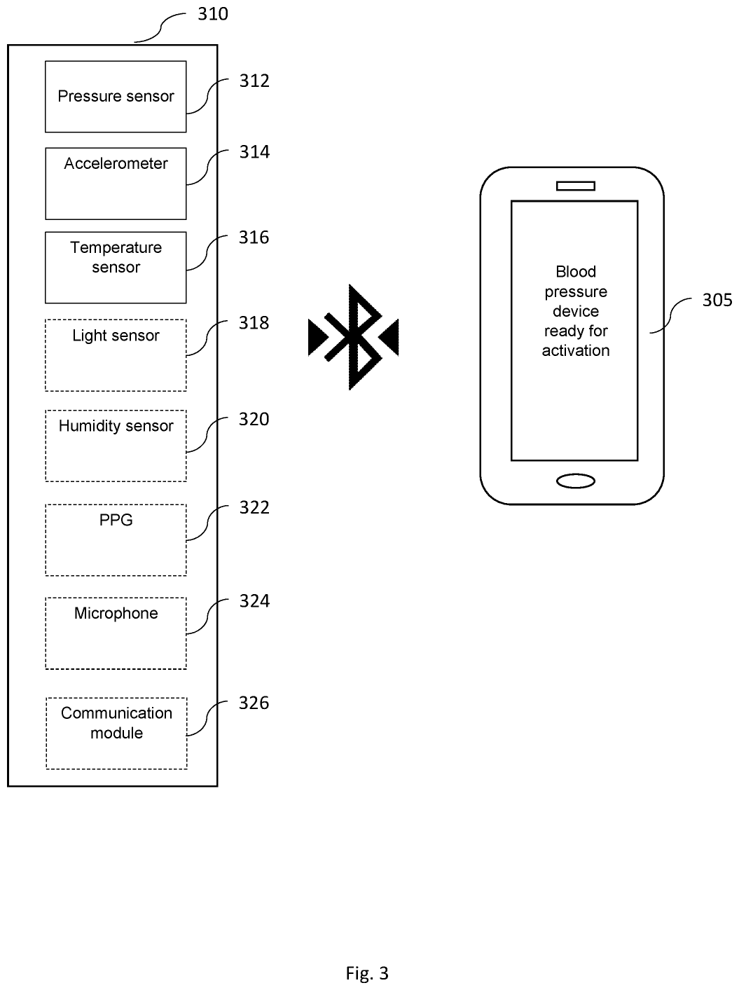

Reference is now made to FIG. 3, which schematically depicts a block diagram of a device 310 for monitoring blood pressure with subject awareness information. Device 310 is operable by a mobile device 305 application, according to an exemplary embodiment of the current invention. Device 310, which may include a wearable device, such as, but not limited to, a wrist/hand/leg/ankle band, includes a pressure sensor 312, an accelerometer 314 and a temperature sensor 316 and may also include a light sensor 318, a humidity sensor 320, PPG (photoplethysmography) sensor 322 and/or a microphone 324.

Pressure sensor 312 is configured to directly sense pressure at a peripheral artery, in the vicinity of which device 310 is attached. The peripheral artery may include a radial, ulnar and/or brachial artery for the arm and femoral, popliteal, tibial, and/or fibular artery of the leg of the subject being monitored. Accelerometer 314 temperature sensor 316, light sensor 318, (skin) humidity sensor 320, PPG sensor 322 and microphone 324 are configured to provide signals indicative of the physiologic and/or environmental (awareness) status of the monitored subject. Signals from all the above-mentioned sensors or any other relevant sensors may by transmitted to mobile device 305 or to any other location (e.g., remote processing unit) by a communication module 326. Communication module 326 may utilize Wi-Fi communication, NFC (Near-field) communication, cellular communication, Bluetooth communication or any other type of communication. Mobile device 305, or any other processing unit, may then process the signals and provide validated (optionally adjusted) blood pressure values, compute contextual blood pressure data, and provide diagnosis, predictions and/or alerts as disclosed herein.

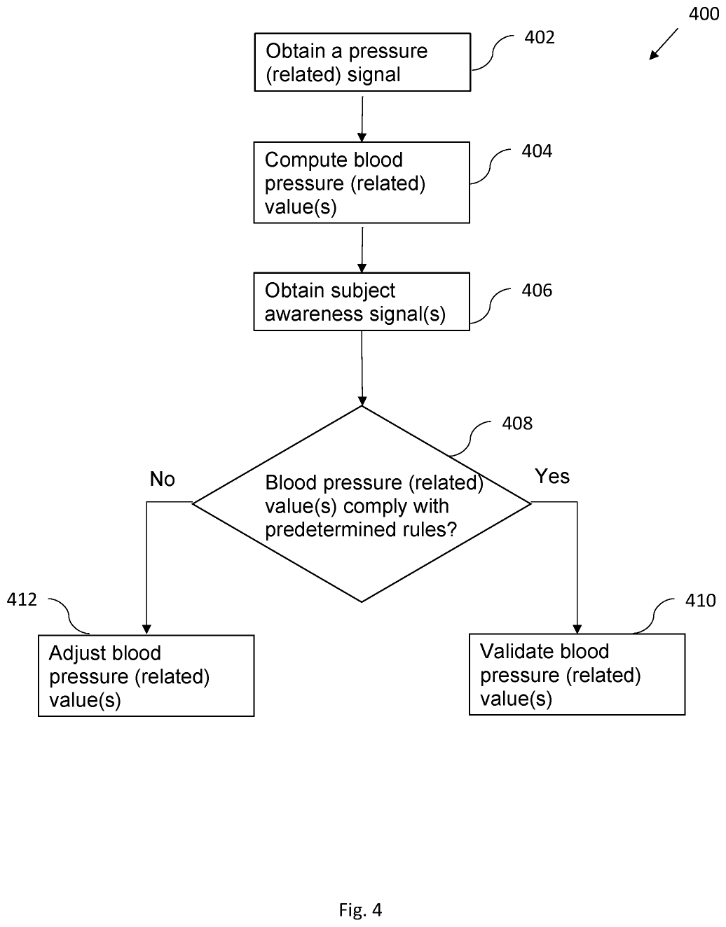

Reference is now made to FIG. 4, which schematically depicts a flow chart 400 of a method for monitoring blood pressure with subject awareness information, according to an exemplary embodiment of the current invention.

Step 402 includes obtaining a pressure signal or a pressure related signal from a pressure sensor which directly senses pressure at a peripheral artery, such as at a radial, ulnar and/or brachial artery for the arm and femoral, popliteal, tibial, and/or fibular artery of the leg of the subject being monitored.

Step 404 includes computing blood pressure value(s), such as a blood pressure waveform, systolic, diastolic and/or mean blood pressure value, or blood pressure related value(s) based on the pressure signal or the pressure related signals obtained in step 402.

Step 406 includes obtaining subject awareness signal(s) related to the subject's present and/or past (historic) surroundings. Such signals may be obtained from subject awareness sensors, such as, but not limited to, accelerometer, gyroscope, magnetometer (compass), steps counter, GPS, barometer, temperature sensor, ambient light sensor (light level), microphone (noise level and speech recognition), humidity sensor, impedance sensor or any combination thereof.

Step 408 includes determining (using a processing unit) whether the blood pressure (related) value(s) computed in step 404 comply with certain requirements (e.g., predetermined blood pressure measurement rules, such as blood pressure measurement guidelines of the ACC/AHA) concerning the subject's posture, activity, surroundings, etc. during or before blood pressure measurements. This determination is based on an analysis of the subject awareness signal(s) obtained in step 406.

If the blood pressure value(s) (such as waveform) comply with the predetermined rules, the blood pressure value(s) are validated (Step 410). If, on the other hand, the blood pressure value(s) (such as waveform) do not comply with predetermined rules, the blood pressure value(s) is adjusted accordingly (Step 412).

The blood pressure adjustment step 412 may include adjustment of blood pressure values based on combining identification of the state of the subject with a method/calculation/or function that can adjust/transfer/translate these BP values to corresponding BP values taken as if the subject state was according to the guidelines. In some embodiments, the BP values adjustment may be done using methods/calculations/functions based on one or more of the following: physiological modeling, statistical methods (e.g., statistical correlation), and machine learning.

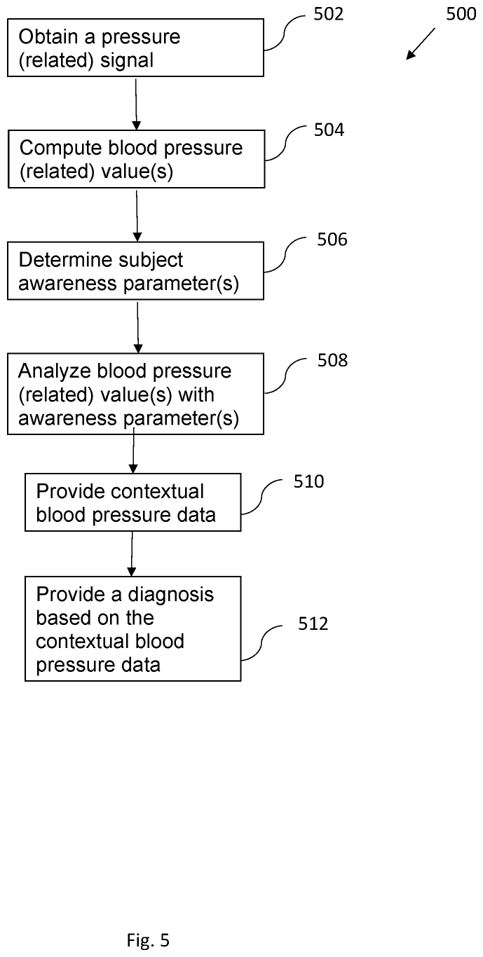

Reference is now made to FIG. 5, which schematically depicts a flow chart 500 of a method for monitoring, analyzing and diagnosing blood pressure related conditions, according to an exemplary embodiment of the current invention.

Step 502 includes obtaining a pressure signal or a pressure related signal from a pressure sensor which directly senses pressure at a peripheral artery, such as at a radial, ulnar and/or brachial artery for the arm and femoral, popliteal, tibial, and/or fibular artery of the leg of the subject being monitored.

Step 504 includes computing blood pressure value(s), such as a blood pressure waveform, or blood pressure related value(s) based on the pressure signal or the pressure related signals obtained in step 502.

Step 506 includes determining subject awareness parameter(s). The subject awareness parameters may be related to the subject's present and/or past (historic) surroundings, for example, altitude, location, place, weather, local time, light level, surrounding noise type and/or level, level of crowdedness, traffic status or any combination thereof. Such parameters may be determined by analyzing signals obtained from subject awareness sensors, such as, but not limited to, accelerometer, gyroscope, magnetometer (compass), steps counter, GPS, barometer, temperature sensor, ambient light sensor (light level), microphone (noise level and speech recognition), humidity sensor, impedance sensor or any combination thereof.

Step 508 includes analyzing the blood pressure (related) value(s) obtained in Step 504 in the context of the awareness parameter(s) determined in Step 506. This analysis yields contextual blood pressure data provided in Step 510. Contextual blood pressure data correlates the blood pressure value (e.g., waveform) with one or more awareness parameter that the subject is/was experiencing during or before blood pressure monitoring, which may affect the measurement.

Step 512 includes providing a diagnosis based on the contextual blood pressure data. The diagnosis relates to blood pressure, cardiac activity and/or related disorder. For example, high blood pressure, high blood pressure variability, white coat syndrome, sleep apnea, aortic valve regurgitation (Pulsus bisferiens), Pulsus alternans and/or left ventricular impairment, Pulsus paradoxus, and Pre-eclampsia.

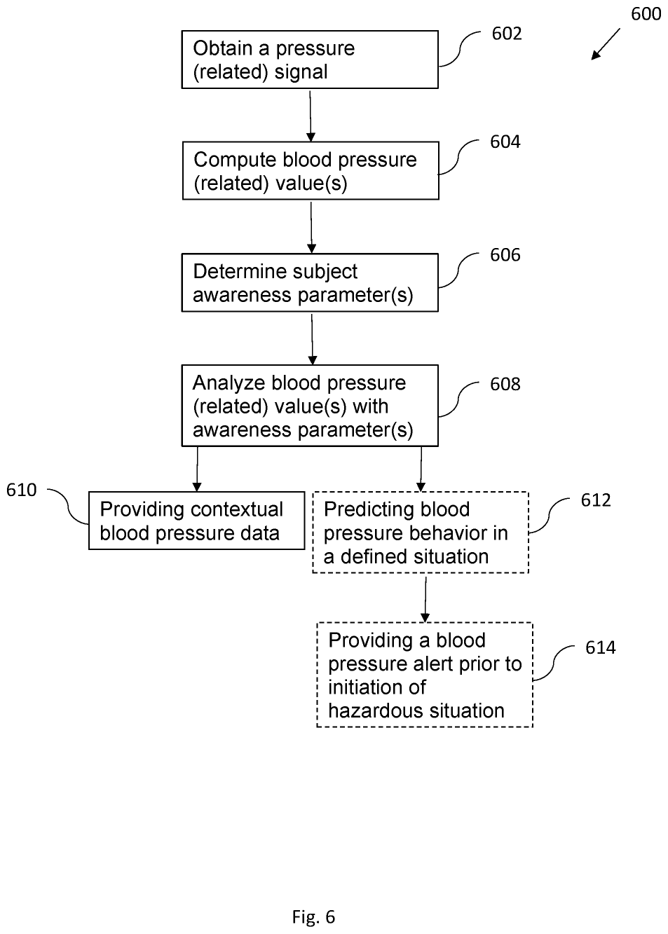

Reference is now made to FIG. 6, which schematically depicts a flow chart 600 of a method for monitoring, analyzing and predicting a blood pressure related condition, according to an exemplary embodiment of the current invention.

Step 602 includes obtaining a pressure signal or a pressure related signal from a pressure sensor which directly senses pressure at a peripheral artery, such as at a radial, ulnar and/or brachial artery for the arm and femoral, popliteal, tibial, and/or fibular artery of the leg of the subject being monitored.

Step 604 includes computing blood pressure value(s), such as a blood pressure waveform, or blood pressure related value(s) based on the pressure signal or the pressure related signals obtained in step 602.

Step 606 includes determining subject awareness parameter(s). The subject awareness parameters may be related to the subject's present and/or past (historic) surroundings, for example, altitude, location, place, weather, local time, light level, surrounding noise type and/or level, level of crowdedness, traffic status or any combination thereof. Such parameters may be determined by analyzing signals obtained from subject awareness sensors, such as, but not limited to, accelerometer, gyroscope, magnetometer (compass), steps counter, GPS, barometer, temperature sensor, ambient light sensor (light level), microphone (noise level and speech recognition), humidity sensor, impedance sensor or any combination thereof.

Step 608 includes analyzing the blood pressure (related) value(s) obtained in Step 604 in the context of the awareness parameter(s) determined in Step 606. This analysis yields contextual blood pressure data provided in Step 610. Contextual blood pressure data correlates the blood pressure value (e.g., waveform) with one or more awareness parameter that the subject is/was experiencing during or before blood pressure monitoring, which may affect the measurement.

The analysis of Step 608 may identify correlations between the blood pressure value (e.g., waveform) and the awareness parameters. Such correlations may allow utilizing machine learning algorithms, learning about the subject's habits based on the one or more correlations and predicting the subject's blood pressure behavior in a defined situation—Step 612. An alarm may then be triggered (Step 614) prior to initiation of a situation which may affect the blood pressure of the subject in a hazardous way.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

In the description and claims of the application, each of the words “comprise” “include” and “have”, and forms thereof, are not necessarily limited to members in a list with which the words may be associated.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What we claim is:

1. A method for measuring blood pressure of a subject, the method comprising:
obtaining, from a pressure sensor, a signal representing a waveform of the blood pressure of the subject;
computing one or more blood pressure values and/or blood pressure related values;
obtaining, from one or more subject awareness sensors and/or medical or non-medical user sources, signal(s) indicative of one or more subject awareness parameters and/or one or more physiologic parameters of the subject;
validating the one or more blood pressure values by determining whether the one or more subject awareness parameters and/or the one or more physiologic parameters of the subject comply with blood pressure measurement rules; and
adjusting the one or more computed blood pressure values and/or blood pressure related values, as if the blood pressure measurement complied with the rules, if at least one of the one or more subject awareness parameters and/or the one or more physiologic parameters of the subject does not comply with the rules.

2. The method of claim 1, further comprising measuring the one or more subject awareness parameters, utilizing the one or more subject awareness sensors, before, during and/or after measuring the blood pressure waveform utilizing the pressure sensor.

3. The method of claim 1, wherein the one or more subject awareness sensors comprise accelerometer, gyroscope, magnetometer, steps counter, GPS, barometer, temperature sensor, ambient light sensor, microphone, humidity sensor, impedance sensor or any combination thereof.

4. The method of claim 1, wherein the one or more subject awareness parameters comprise one or more parameters related to the subject's present and/or past surrounding.

5. The method of claim 1, wherein the one or more subject awareness parameters comprise altitude, location, place, weather, local time, light level, surrounding noise type and/or level, level of crowdedness, traffic status, or any combination thereof.

6. The method of claim 1, further comprising measuring the one or more physiologic parameters of the subject, utilizing one or more sensors, before, during and/or after measuring the blood pressure waveform utilizing the pressure sensor.

7. The method of claim 1, wherein the one or more physiologic parameters comprise one or more present and/or past physiologic parameters selected from the group consisting of: the subject's activity and/or length/intensity thereof, orientation, posture, sleep vs. awake, heart rate, respiration rate, skin humidity/sweat level, or any combination thereof.

8. The method of claim 1, wherein the one or more medical and non-medical user sources comprise a health App, a social platform, a calendar, a fitness App, a communication App or any combination thereof.

9. The method of claim 1, wherein the one or more computed blood pressure values comprise Systolic, Diastolic, Mean, momentary arterial blood pressure, or any combination thereof; and/or wherein the one or more computed blood pressure related values comprise heart rate and/or breathing rate.

10. The method claim 1, wherein the blood pressure measurement rules comprise blood pressure regulatory guidelines.

11. The method of claim 1, wherein the blood pressure measurement rules comprise awake and sleep rules; temporal rules, spatial rules, geographic rules, or any combination thereof.

12. The method of claim 1, wherein the pressure sensor is configured to directly sense pressure at a peripheral artery of the subject.

13. A method for contextual blood pressure analysis, the method comprising:
obtaining, from a wearable pressure sensor, a signal representing a waveform of the blood pressure of the subject;
computing one or more blood pressure values and/or blood pressure related values;
obtaining, from one or more subject awareness sensors and/or medical or non-medical user sources, signal(s) indicative of one or more subject awareness parameters and/or one or more physiologic parameters of the subject;
analyzing the one or more computed blood pressure values and/or blood pressure related values with the one or more subject awareness parameters and/or the one or more physiologic parameters; and
providing contextual blood pressure data.

14. The method of claim 13, wherein the contextual blood pressure data comprises a circadian pattern of blood pressure values along with respective subject awareness parameters.

15. The method of claim 13, further comprising identifying one or more correlations between the blood pressure values and the one or more subject awareness parameters.

16. The method of claim 15, further comprising, providing, based on the one or more correlations, a diagnosis related to blood pressure, cardiac activity and/or related disorder.

17. The method of any one of claim 15, further comprising, based on the one or more correlations, identifying a hazardous situation.

18. The method of claim 17, further comprising providing a blood pressure alert prior to initiation of the hazardous situation.

19. The method of claim 15, further comprising, utilizing machine learning algorithms, learning one or more of the subject's habits based on the one or more correlations, and predicting the subject's blood pressure behavior in a defined situation.

20. The method of claim 13, further comprising measuring the one or more subject awareness parameters, utilizing the one or more subject awareness sensors, before, during and/or after measuring the blood pressure waveform utilizing the pressure sensor.

21. The method of claim 13, wherein the one or more computed blood pressure values comprise Systolic, Diastolic, Mean, momentary arterial blood pressure or any combination thereof; and/or wherein the one or more computed blood pressure related values comprise heart rate and/or breathing rate.

22. The method of claim 13, wherein the one or more physiologic parameters comprise one or more present and/or past (historic) physiologic parameters selected from the group consisting of: the subject's activity and/or length/intensity thereof, orientation, posture, sleep vs. awake, heart rate, respiration rate, skin humidity/sweat level, or any combination thereof.

23. The method of claim 13, wherein the one or more medical and non-medical user sources comprise health Apps, social platforms, calendars, fitness Apps, communication Apps or any combination thereof.

24. The method of claim 13, wherein the pressure sensor is configured to directly sense pressure at a peripheral artery of the subject.

25. A system for measuring blood pressure of a subject, the system comprising:
a pressure sensor configured to sense pressure at a peripheral artery of the subject and to provide a signal representing a waveform of the blood pressure; and
electric circuitry and associated software/firmware/computation component/algorithm configured to:
compute one or more blood pressure values and/or blood pressure related values based on the signal representing a waveform of the blood pressure;
obtain, from one or more subject awareness sensors and/or medical or non-medical user sources, signal(s) indicative of one or more subject awareness parameters and/or one or more physiologic parameters of the subject;
validate the one or more blood pressure values by determining whether the one or more subject awareness parameters and/or the one or more physiologic parameters of the subject comply with blood pressure measurement rules; and
adjusting the one or more computed blood pressure values and/or blood pressure related values, as if the blood pressure measurement complied with the rules, if at least one of the one or more subject awareness parameters and/or the one or more physiologic parameters of the subject does not comply with the rules.

26. A system for contextual blood pressure analysis, the system comprising:
a wearable pressure sensor configured to directly sense pressure at a peripheral artery of a subject and to provide a signal representing a waveform of the blood pressure; and
electric circuitry and associated software/firmware/computation component/algorithm configured to:
compute one or more blood pressure values and/or blood pressure related values based on the signal representing a waveform of the blood pressure;
obtain, from one or more subject awareness sensors and/or medical or non-medical user sources, signal(s) indicative of one or more subject awareness parameters and/or one or more physiologic parameters of the subject;
analyze the one or more computed blood pressure values and/or blood pressure related values with the one or more subject awareness parameters and/or the one or more physiologic parameters; and
provide contextual blood pressure data.
